SEQUENCE LISTING (32 sequences)

SEQ ID NO: 1 - protein, 5 aa, artificial sequence
  anti-ILDR2/no.1 HCDR1
SYAIS

SEQ ID NO: 2 - protein, 17 aa, artificial sequence
  anti-ILDR2/no.1 HCDR2
GIIPILGIANYAQKFQG

SEQ ID NO: 3 - protein, 13 aa, artificial sequence
  anti-ILDR2/no.1 HCDR3
ARGRLPYGDFWDS

SEQ ID NO: 4 - protein, 16 aa, artificial sequence
  anti-ILDR2/no.1 LCDR1
RSSQSLLYSNGYNYLD

SEQ ID NO: 5 - protein, 7 aa, artificial sequence
  anti-ILDR2/no.1 LCDR2
LGSNRAS

SEQ ID NO: 6 - protein, 9 aa, artificial sequence
  anti-ILDR2/no.1 LCDR3
MQALQTPLT

SEQ ID NO: 7 - protein, 120 aa, artificial sequence
  anti-ILDR2/no.1 heavy chain VD
QVQLVQSGAEVKKPGSSVKVSCKASGGTFSSYAISWVRQAPGQGLEWMGGIIPILGIANYAQKFQGRVTITADKSTSTAYMELSSLRSEDTAVYYCARGRLPYGDFWDSWGQGTLVTVSS

SEQ ID NO: 8 - protein, 112 aa, artificial sequence
  anti-ILDR2/no.1 light chain VD
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQALQTPLTFGGGTKLEIR

SEQ ID NO: 9 - protein, 445 aa, artificial sequence
  anti-ILDR2/no.1 heavy chain
QVQLVQSGAEVKKPGSSVKVSCKASGGTFSSYAISWVRQAPGQGLEWMGGIIPILGIANYAQKFQGRVTITADKSTSTAYMELSSLRSEDTAVYYCARGRLPYGDFWDSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 10 - protein, 219 aa, artificial sequence
  anti-ILDR2/no.1 light chain
DIVMTQSPLSLPVTPGEPASISCRSSQSLLYSNGYNYLDWYLQKPGQSPQLLIYLGSNRASGVPDRFSGSGSGTDFTLKISRVEAEDVGVYYCMQALQTPLTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 120 aa, artificial sequence [sequence omitted]

SEQ ID NO: 12 - protein, 5 aa, artificial sequence
  Pembrolizumab HCDR1
NYYMY

SEQ ID NO: 13 - protein, 17 aa, artificial sequence
  Pembrolizumab HCDR2
GINPSNGGTNFNEKFKN

SEQ ID NO: 14 - protein, 11 aa, artificial sequence
  Pembrolizumab HCDR3
RDYRFDMGFDY

SEQ ID NO: 15 - protein, 111 aa, artificial sequence [sequence omitted]

SEQ ID NO: 16 - protein, 15 aa, artificial sequence
  Pembrolizumab LCDR1
RASKGVSTSGYSYLH

SEQ ID NO: 17 - protein, 7 aa, artificial sequence
  Pembrolizumab LCDR2
LASYLES

SEQ ID NO: 18 - protein, 9 aa, artificial sequence
  Pembrolizumab LCDR3
QHSRDLPLT

SEQ ID NO: 19 - protein, 447 aa, artificial sequence
  Pembrolizumab Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 218 aa, artificial sequence
  Pembrolizumab Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 113 aa, artificial sequence [sequence omitted]

SEQ ID NO: 22 - protein, 5 aa, artificial sequence
  Nivolumab HCDR1
NSGMH

SEQ ID NO: 23 - protein, 17 aa, artificial sequence
  Nivolumab HCDR2
VIWYDGSKRYYADSVKG

SEQ ID NO: 24 - protein, 4 aa, artificial sequence
  Nivolumab HCDR3
NDDY

SEQ ID NO: 25 - protein, 107 aa, artificial sequence [sequence omitted]

SEQ ID NO: 26 - protein, 11 aa, artificial sequence
  Nivolumab LCDR1
RASQSVSSYLA

SEQ ID NO: 27 - protein, 7 aa, artificial sequence
  Nivolumab LCDR2
DASNRAT

SEQ ID NO: 28 - protein, 9 aa, artificial sequence
  Nivolumab LCDR3
QQSSNWPRT

SEQ ID NO: 29 - protein, 440 aa, artificial sequence
  Nivolumab Heavy Chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 214 aa, artificial sequence
  Nivolumab Light Chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 453 aa, artificial sequence
  TPP-75 Isotype control HC
QVELLESGGGLVQPGGSLRLSCAASGFTFSSYAMSWVRQAPGKGLEWVSAISGSGGSTYYADSVKGRFTISRDNSKNTLYLQMNSLRAEDTAVYYCARGVGKAHRFGVVPRGGMDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 216 aa, artificial sequence
  TPP-75 Isotype control LC
DIVLTQPPSASGTPGQRVTISCSGSSSNIGSNTVNWYQQLPGTAPKLLIYGNSNRPSGVPDRFSGSKSGTSASLAISGLRSEDEADYYCAAWDDSLNGVLFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2022/0169736 A1
(43) Pub. Date: Jun. 2, 2022

(54) COMBINATIONS OF ANTI-ILDR2 ANTIBODIES AND PD-1 ANTAGONISTS

(71) Applicants: Bayer Aktiengesellschaft, Leverkusen (DE); Compugen LTD., Holon (IL)

(72) Inventors: Lars RÖSE, Berlin (DE); Uwe GRITZAN, Köln (DE); Spencer LIANG, San Mateo, CA (US); Andrew POW, San Diego, CA (US); John HUNTER, Piedmont, CA (US); Ofer LEVY, Moshav Mesilat Zion (IL); Ilan VAKNIN, Modiin-Maccabim-Reut (IL)

(73) Assignees: Bayer Aktiengesellschaft, Leverkusen (DE); Compugen LTD., Holon (IL)

(21) Appl. No.: 17/594,269

(22) PCT Filed: Apr. 6, 2020
(86) PCT No.: PCT/EP2020/059745

§ 371 Date: Oct. 8, 2021

Related U.S. Application Data
(60) Provisional application No. 62/832,320, filed on Apr. 11, 2019.

(51) Int. Cl. C07K 16/28; A61P 35/00

(52) U.S. Cl. CPC C07K 16/2827; A61P 35/00; C07K 16/2818

(57) ABSTRACT

The present invention relates to an anti-ILDR2 antibody, a fragment or derivative thereof, a modified antibody format, or an antibody mimetic for use in combination with a PD-1 antagonist in the treatment of cancer. Other aspects of the present invention relate to a combination comprising an anti-ILDR2 antibody and a PD-1 antagonist and the use of such combination as a medicament, as well as methods of treatment or prophylaxis of a cancer in a subject, comprising administering to said subject a therapeutically effective amount of the antibodies as described herein. Further, the present invention relates to a kit comprising anti-ILDR2 antibodies and PD-1 antagonists and optionally one or more further pharmaceutical agents.

### FIELD OF THE INVENTION

The present invention relates to an anti-ILDR2 antibody, a fragment or derivative thereof, a modified antibody format, or an antibody mimetic for use in combination with a PD-1 antagonist in the treatment of cancer. Other aspects of the present invention relate to a combination comprising an anti-ILDR2 antibody and a PD-1 antagonist and the use of such combination as a medicament, as well as methods of treatment or prophylaxis of a cancer in a subject, comprising administering to said subject a therapeutically effective amount of the antibodies as described herein. Further, the present invention relates to a kit comprising anti-ILDR2 antibodies and PD-1 antagonists and optionally one or more further pharmaceutical agents.

### BACKGROUND

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like escape from anti-tumor immune response, enhanced survival/resistance towards apoptosis and a limitless proliferative potential. Thus, it is preferred to develop drugs for cancer therapy addressing distinct features of established tumors such as—but not limited to—immunosuppressive tumor microenvironment (TME) and insufficient T cell priming

The B7 family of immune-regulatory ligands consists of structurally related, cell-surface protein ligands, which bind to receptors on lymphocytes that regulate immune responses.

The activation of T and B lymphocytes is initiated by engagement of cell-surface, antigen-specific T cell receptors or B cell receptors, but additional signals delivered simultaneously by B7 ligands determine the ultimate immune response. These ‘costimulatory’ or ‘coinhibitory’ signals are delivered by B7 ligands through the CD28 family of receptors on lymphocytes.

The family of B7 proteins includes: B7.1 (CD80), B7.2 (CD86), inducible costimulator ligand (ICOS-L), programmed death-1 ligand (PD-L1, also called B7-1)), programmed death-2 ligand (PD-L2), B7-H3, and B7-H4. Members of the family have been characterized predominantly in humans and mice, but some members are also found in birds. They share 20-40% amino-acid identity and are structurally related, with the extracellular domain containing tandem domains related to variable and constant immunoglobulin domains. B7 ligands are expressed in lymphoid and non-lymphoid tissues. The importance of the family in regulating immune responses is shown by the development of immunodeficiency and autoimmune diseases in mice with mutations in B7-family genes. Manipulation of the signals delivered by B7 ligands has shown potential in the treatment of autoimmunity, inflammatory diseases and cancer.

The interaction of B7-family members with their respective costimulatory receptor, usually a member of the CD28-related family, augments immune responses, while interaction with co-inhibitory receptors, such as CTLA4, attenuates immune responses.

Clearly, each B7 molecule has developed its own niche in the immune system. As specific niches of B7 family members continue to be dissected, their diagnostic and therapeutic potential becomes ever more apparent. Many of the B7 superfamily members were initially characterized as T cell co-stimulatory molecules. However, more recently it has become clear they can also co-inhibit T cell responses. Thus, B7 family members may have opposing effects on an immune response.

Members of the B7 family have become targets for immune checkpoint inhibitor therapy.

Recently, the PD-1/PD-L1 signalling pathway has emerged as an important regulator of the activity of the immune system. In cancer, tumor cells express PD-L1, the ligand of PD-1, by which they can evade their killing by the host immune system. Inhibitors against PD-1 and its ligands PD-L1 and PD-L2 have recently been developed which interfere with this immune-suppressive mechanism and have shown amazing clinical efficacy, by extension of the overall survival of patients with various types of cancer. Some of these inhibitors have been approved for various cancer indications such as melanoma, NSCLC, HNSCC, RCC, bladder cancer and NHL. A large number of additional clinical trials are in progress in other indications and/or in combination with a variety of other antitumor agents in order to improve the therapeutic activity.

PD-1 inhibitors are biologics, primarily immunoglobulins of the G subclass, which bind to programmed cell death protein 1 also known as PD-1 and block its activity. Known PD-1 inhibitors are nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine).

PD-1 (also known as CD279) is a receptor protein which is expressed as monomer on the surface of various immune cells mainly on activated CD4⁺ and CD8⁺ T cells, on macrophages and on activated B cells, but was also found on natural killer (NK) cells and antigen presenting cells (APC). Upon binding to its ligand PD-L1 or PD-L2, the phosphatase SHP-2 is recruited which dephosphorylates the kinase ZAP70, a major component of the T cell receptor (TCR) signaling complex. This shuts down TCR signaling and inhibits the cytotoxic activity of the T cells, their interferon gamma production and proliferation. In addition, PD-1 ligation up-regulates E3-ubiquitin ligases CBL-b and c-CBL that trigger T cell receptor down-modulation. PD-1 is encoded by the Pdcd1 gene in humans and is transcriptionally activated by transcription factors NFATc1, IRF9 and FoxO1, which are activated upon TCR activation and by T cell exhaustion signals such as transforming growth factor β and eomesodermin. The activation induced expression of PD-1 suggests that this receptor regulates rather the later phase of the immune response in the peripheral tissue (effector phase, memory response and chronic infection).

However, despite the great success of the above identified approaches, it has turned out that some of them are either not sustainable in their efficacy, i.e., a recurrence of the disease occurs, and/or are only efficacious in some types of cancers.

Therefore, there is a great need in the field of immune checkpoint inhibitor therapy for providing new and improved therapies as well as for improving existing therapies.

The recently identified ILDR2 (Immunoglobulin Like Domain Containing Receptor 2), also known as C1ORF32, is a novel member of the B7/CD28 family. ILDR2 comprises an IgV domain; in addition of it being a type I membrane protein, like other known B7 members—which eventually gave rise to its annotation to the B7 family. Also, two alternatively spliced variants of ILDR2 (H19011-1-P8 and H19011-1-P9), which share only the first 5 exons with the wild type C1ORF32 are similar to the known B7 family members in their exons' sizes and the position of the IgV and transmembrane domains within these exons. For a thorough characterization of ILDR2, see WO2009032845, the content of which is incorporated by reference herein.

Thus far, no therapies targeting this recently identified receptor have been developed. It is hence one object of the present invention to provide new and improved immune checkpoint inhibitor therapies targeting ILDR2. Especially the combination possibilities with already existing immune checkpoint inhibitor therapies and their improvement in order to overcome the above stated drawbacks of the currently existing immune checkpoint inhibitor therapies with new combinations are being investigated.

### SUMMARY OF THE INVENTION

The present invention therefore provides for an anti-ILDR2 antibody, a fragment or derivative thereof, a modified antibody format, or an antibody mimetic for use in combination with a PD-1 antagonist in the treatment of cancer, wherein the anti-ILDR2 antibody, a fragment or derivative thereof, a modified antibody format, or an antibody mimetic further comprises at least the three CDR heavy chain sequences according to SEQ ID NO.1, SEQ ID NO.2 and SEQ ID NO.3 and the three CDR light chain sequences according to SEQ ID NO.4, SEQ ID NO.5 and SEQ ID NO.6.

In one embodiment of present invention the anti-ILDR2 antibody, fragment or derivative thereof, a modified antibody format or an antibody mimetic comprises at least one heavy chain variable region sequence that is at least 95%, at least 96%, at least 97%, at least 98%, at least 99%, or 100% identical to the sequence of SEQ ID NO.7 and/or at least one light chain variable region sequence that is at least 95%, at least 96%, at least 97%, at least 98%, at least 99%, or 100% identical to the sequence of SEQ ID NO.8.

In a further embodiment of present invention the anti-ILDR2 antibody, fragment or derivative thereof, a modified antibody format or an antibody mimetic comprises at least one heavy chain sequence that is at least 95%, at least 96%, at least 97%, at least 98%, at least 99%, or 100% identical to the sequence of SEQ ID NO.9; and/or at least one light chain sequence that is at least 95%, at least 96%, at least 97%, at least 98%, at least 99%, or 100% identical to the sequence of SEQ ID NO.10.

In one embodiment of present invention the PD-1 antagonist is an antibody, a fragment or derivative thereof, a modified antibody format, or an antibody mimetic, all of which having PD-1 binding properties.

In a further embodiment of present invention the PD-1 antagonist is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine).

In a preferred embodiment of present invention the PD-1 antagonist is nivolumab (Opdivo, BMS-936558, MDX1106) or pembrolizumab (Keytruda, MK-3475, lambrolizumab), most preferred is pembrolizumab (Keytruda, MK-3475, lambrolizumab).

In one embodiment of present invention the PD-1 antagonist 1 comprises

i) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or

ii) at least one heavy chain sequence that is at least 95%, at least 96%, at least 97%, at least 98%, at least 99%, or 100% identical to the sequence of SEQ ID NO.19; and/or

iii) at least one light chain sequence that is at least 95%, at least 96%, at least 97%, at least 98%, at least 99%, or 100% identical to the sequence of SEQ ID NO.20.

In a further embodiment the present invention provides for an anti-ILDR2 antibody, a fragment or derivative thereof, a modified antibody format, or an antibody mimetic as defined herein for use in combination with a PD-1 antagonist as defined herein in the treatment of cancer, wherein at least one of the anti-ILDR2 antibody and the PD-1 antagonist is administered in simultaneous, separate, or sequential combination with one or more pharmaceutical agents.

The present invention furthermore provides for a novel combination comprising at least two components, component A and component B, wherein component A and component B are administered simultaneously, concurrently, separately or sequentially, wherein component A is an anti-ILDR2 antibody, a fragment or derivative thereof, a modified antibody format, or an antibody mimetic as defined herein, and wherein component B is a PD-1 antagonist as defined herein.

One embodiment of present invention relates to the combination as defined herein for use as a medicament.

A further embodiment relates to the combination as described herein for use in the treatment or prophylaxis of a neoplastic disease, such as cancer, or an immune disease or disorder, wherein the combination is administered in one or more therapeutically efficient dosages.

Another embodiment of present invention relates to a method for treating a patient suffering from a neoplastic disease, such as cancer, comprising administering to said patient an anti-ILDR2 antibody, a fragment or derivative thereof, a modified antibody format, or an antibody mimetic as described herein and a PD-1 antagonist as defined herein, in one or more therapeutically efficient dosages, wherein the anti-ILDR2 antibody and the PD-1 antagonist are administered simultaneously, concurrently, separately or sequentially.

Another embodiment of present invention relates to the use of an anti-ILDR2 antibody, a fragment or derivative thereof, a modified antibody format, or an antibody mimetic as defined herein, and a PD-1 antagonist as defined herein for the manufacture of a medicament for the treatment of cancer.

A further embodiment of present invention relates to a kit comprising an anti-ILDR2 antibody, a fragment or derivative thereof, a modified antibody format, or an antibody mimetic as described herein, a PD-1 antagonist as described herein and one or more further pharmaceutical agents.

### BRIEF DESCRIPTION OF THE FIGURES

FIG. 1: Delay of tumor growth by a combination of aPD-1 and aILDR2/no. 1 in the CT26 tumor model.

The terms “rIgG” and “hIgG” refer to rat and human immunoglobulin G, respectively.

The term “aPD-1” refers to anti-PD-1 monoclonal antibody RMP1-14 that reacts with mouse PD-1 (programmed death-1), also known as CD279, and that blocks the interaction of PD-L1 and PD-L2 with PD-1.

The term “aILDR2/no. 1” refers to the anti-ILDR2 antibody according to present invention as further described in the detailed description herein under.

The term “isotype control” refers to the use of a monoclonal antibody of the same isotype, same species, but directed against an irrelevant antigen. The isotype controls used for the experiments in present invention are antibody TPP-75 as disclosed in SEQ ID NOs.31 and 32 (isotype control for the anti-ILDR2 antibody) and InVivoPlus rat IgG2a isotype control, anti-trinitrophenol, clone 2A3, catalog #BP0089 from BioXcell (isotype control for the anti-PD-1 antibody).

In the CT26 tumor model no monotherapy efficacy was observed vs. isotype control with treatment using anti-ILDR2 antibody alone. Treatment with aPD-1 alone showed tumor reducing activity. However, a combination of aPD-1 and aILDR2/no.1 synergistically delayed tumor growth statistically significant compared to isotype control and compared to aPD-1 monotherapy. Significance of monotherapy and combination treatment vs. isotype control (p=0,0001) or aPD-1 (p=0,0291) as determined by One way ANOVA analysis of final logarithmized data points (Dunnett) Start of treatment: q3d i.p.

FIG. 2: Efficacy of therapeutic interventions according to the present invention in a CT26 syngeneic mouse model. Activity is measured as tumor volume under treatment with the therapeutic relative to tumor volume under treatment with the isotype control as defined above.

### DEFINITIONS

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent with the meaning commonly understood in the art. Such references include, but are not limited to, Singleton et al., Dictionary of Microbiology and Molecular Biology (2nd ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resource available to the person of ordinary skill in the art providing definitions of terms used herein having the meaning commonly understood in the art can be consulted. For the purposes of the present invention, the following terms are further defined. Additional terms are defined elsewhere in the description. As used herein and in the appended claims, the singular forms “a,” and “the” include plural reference unless the context clearly dictates otherwise.

“Amino acids” may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as an ILDR2 antagonist of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule (or, if necessary, undergo affinity maturation or deiimuization), and including dual specific, bispecific, multispecific, and dual variable domain immunoglobulins. Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype. In one embodiment of present invention, the anti ILDR2 antibody is fully human and of the IgG2 subclass.

An “antibody-based binding protein”, as used herein, may represent any protein that contains at least one antibody-derived V_{H}, V_{L}, or C_{H} immunoglobulin domain in the context of other non-immunoglobulin, or non-antibody derived components. Such antibody-based proteins include, but are not limited to (i) F_{c}-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (V_{L}), variable heavy (V_{H}), constant light (C_{L}) and constant heavy 1 (C_{H}1) domains; (ii) a F(ab′)2 fragment, which is a bivalent fragment comprising two Fab fragments linked by a disulfide bridge at the hinge region; (iii) a heavy chain portion of a F_{ab} (F_{d}) fragment, which consists of the V_{H} and C_{H}1 domains; (iv) a variable fragment (Fᵥ) fragment, which consists of the V_{L} and V_{H} domains of a single arm of an antibody, (v) a domain antibody (dAb) fragment, which comprises a single variable domain; (vi) an isolated complementarity determining region (CDR); (vii) a single chain Fᵥ Fragment (scFᵥ); (viii) a diabody, which is a bivalent, bispecific antibody in which V_{H} and V_{L} domains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fᵥ segments (V_{H}-C_{H}1-V_{H}-C_{H}1) which, together with complementarity light chain polypeptides, form a pair of antigen binding regions; and (x) other non-full length portions of immunoglobulin heavy and/or light chains, or mutants, variants, or derivatives thereof, alone or in any combination.

The term “modified antibody format”, as used herein, encompasses antibody-drug-conjugates, Polyalkylene oxide-modified scFv, Monobodies, Diabodies, Camelid Antibodies, Domain Antibodies, bi-or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerised constructs comprising CH3+VL+VH, and the like.

The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, nucleic acid aptamers, artificial antibodies produced by molecular imprinting of polymers, peptide libraries from bacterial genomes, SH-3 domains, stradobodies, “A domains” of membrane receptors stabilised by disulfide bonds and Ca2+, CTLA4-based compounds, Fyn SH3, and aptamers (oligonucleic acid or peptide molecules that bind to a specific target molecules)

The term “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

As used herein “ILDR2” relates to Immunoglobulin Like Domain Containing Receptor 2, also known as C1ORF32, which is a novel member of the B7/CD28 family. For a thorough characterization of ILDR2, see WO2009032845, the content of which is herein incorporated by reference.

The terms “anti-ILDR2 antibody”, “aILDR2”, “aILDR2 antibody” and “an antibody that binds to ILDR2” refer to an antibody that is capable of binding ILDR2 with sufficient affinity such that the antibody is useful as a diagnostic and/or therapeutic agent in targeting ILDR2. In one embodiment, the extent of binding of an anti-ILDR2 antibody to an unrelated, non-ILDR2 protein is less than about 5%, or preferably less than about 2% of the binding of the antibody to ILDR2 as measured, e.g., by a surface plasmon resonance (SPR). In certain embodiments, an antibody that binds to ILDR2 has a dissociation constant (KD) of ≤1 μM, ≤100 nM, ≤10 nM, ≤1 nM, ≤0.1 nM, ≤0.01 nM, or ≤0.001 nM (e.g. 10-8 M or less, e.g. from 10-8 M to 10-13 M, e.g., from 10-9 M to 10-13 M). In certain embodiments, an anti-ILDR2 antibody binds to an epitope of ILDR2 that is conserved among ILDR2 from different species.

The term “PD-1 antagonist” refers to any type of molecule that is capable to block the biological response triggered by PD-1 agonists. PD-1 agonists include the ligands PD-L1 and PD-L2. The antagonist is useful as a therapeutic agent in targeting PD-1.

The terms “anti-PD-1 antibody”, “aPD-1”, “aPD-1 antibody” and “an antibody that binds to PD-1” refer to an antibody that is capable of binding PD-1 with sufficient affinity such that the antibody is useful as a diagnostic and/or therapeutic agent in targeting PD-1.

As used herein, the term “Complementarity Determining Regions” (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; (Kabat et al., Sequences of Proteins of Immulological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA1, and IgA2. A preferred class of immunoglobulins for use in the present invention is IgG.

The heavy-chain constant domains that correspond to the different classes of antibodies are called [alpha], [delta], [epsilon], [gamma], and [mu], respectively. The subunit structures and three-dimensional configurations of different classes of immunoglobulins are well known. As used herein antibodies are conventionally known antibodies and functional fragments thereof.

Variants of the antibodies or antigen-binding antibody fragments contemplated in the invention are molecules in which the binding activity of the antibody or antigen-binding antibody fragment is maintained.

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source). Examples of human antibodies include antibodies as described in Söderlind et al., Nature Biotech. 2000, 18:853-856.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or non-proteinaceous solutes.

As used herein, an antibody “binds specifically to”, is “specific to/for” or “specifically recognizes” an antigen of interest, e.g. a tumor-associated polypeptide antigen target, is one that binds the antigen with sufficient affinity such that the antibody is useful as a therapeutic agent in targeting a cell or tissue expressing the antigen, and does not significantly cross-react with other proteins or does not significantly cross-react with proteins other than orthologues and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent KD for the antigen of less than about 10-4 M, alternatively less than about 10-5 M, alternatively less than about 10-6 M, alternatively less than about 10-7 M, alternatively less than about 10-8 M, alternatively less than about 10-9 M, alternatively less than about 10-10 M, alternatively less than about 10-11 M, alternatively less than about 10-12 M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to surface plasmon resonance (SPR), Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” or “affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “KD” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules Affinity can be measured by common methods known in the art, including those described herein.

As used herein, the term “epitope” includes any protein determinant capable of specific binding to an immunoglobulin or T cell receptor. Epitopic determinants usually consist of chemically active surface groupings of molecules such as amino acids or sugar side chains, or combinations thereof and usually have specific three dimensional structural characteristics, as well as specific charge characteristics.

An “antibody that binds to the same epitope” as a reference antibody or “an antibody which competes for binding” to a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. “Sequence homology” indicates the percentage of amino acids that either is identical or that represent conservative amino acid substitutions.

“Neoplastic diseases” are conditions that cause tumor growth—both benign and malignant. A neoplasm is an abnormal growth of cells, also known as a tumor.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

It is one object of the present invention to provide new and improved immune checkpoint inhibitor therapies targeting ILDR2. Especially the combination possibilities with already existing immune checkpoint inhibitor therapies and their improvement in order to overcome the drawbacks of the currently existing immune checkpoint inhibitor therapies by providing new combination therapies have been investigated.

The present invention therefore relates to an anti-ILDR2 antibody, a fragment or derivative thereof, a modified antibody format, or an antibody mimetic for use in combination with a PD-1 antagonist in the treatment of cancer. Other aspects of the present invention relate to the use of such anti-ILDR2 antibodies in combination with PD-1 antagonists as a medicament, as well as methods of treatment or prophylaxis of a cancer in a subject, comprising administering to said subject a therapeutically effective amount of the antibodies as described herein.

Surprising effects in an in vivo tumor model were observed when administering an anti-ILDR2 antibody and a PD-1 antagonist, both as further defined herein. The therapeutic efficacy of the combination described in the present invention has shown superiority to the efficacy achieved by a PD-1 antagonist or anti-ILDR2 antibody alone.

The present invention therefore provides for an anti-ILDR2 antibody, a fragment or derivative thereof, a modified antibody format, or an antibody mimetic for use in combination with a PD-1 antagonist in the treatment of cancer, wherein the an anti-ILDR2 antibody, a fragment or derivative thereof, a modified antibody format, or an antibody mimetic further comprises at least the three CDR heavy chain sequences according to SEQ ID NO.1, SEQ ID NO.2 and SEQ ID NO.3 and the three CDR light chain sequences according to SEQ ID NO.4, SEQ ID NO.5 and SEQ ID NO.6.

In one embodiment of present invention the anti-ILDR2 antibody, fragment or derivative thereof, a modified antibody format or an antibody mimetic comprises at least one heavy chain variable region sequence that is at least 95%, at least 96%, at least 97%, at least 98%, at least 99%, or 100% identical to the sequence of SEQ ID NO.7 and/or at least one light chain variable region sequence that is at least 95%, at least 96%, at least 97%, at least 98%, at least 99%, or 100% identical to the sequence of SEQ ID No 8.

In a further embodiment of present invention the anti-ILDR2 antibody, fragment or derivative thereof, a modified antibody format or an antibody mimetic, that comprises at least one heavy chain sequence that is at least 95%, at least 96%, at least 97%, at least 98%, at least 99%, or 100% identical to the sequence of SEQ ID NO.9; and/or at least one light chain sequence that is at least 95%, at least 96%, at least 97%, at least 98%, at least 99%, or 100% identical to the sequence of SEQ ID NO.10.

In one embodiment of present invention the PD-1 antagonist is an antibody, a fragment or derivative thereof, a modified antibody format, or an antibody mimetic, all of which having PD-1 binding properties.

In a further embodiment of present invention the PD-1 antagonist is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine).

In a preferred embodiment of present invention the PD-1 antagonist is nivolumab (Opdivo, BMS-936558, MDX1106) or pembrolizumab (Keytruda, MK-3475, lambrolizumab), most preferred is pembrolizumab (Keytruda, MK-3475, lambrolizumab).

“Nivolumab”, developed by Bristol-Myers Squibb, (trade name “OPDIVO”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449). For example it is used as a first line treatment for inoperable or metastatic melanoma in combination with ipilimumab if the cancer does not have a mutation in BRAF, as a second-line treatment following treatment with ipilimumab and if the cancer has a mutation in BRAF, with a BRAF inhibitor, as a second-line treatment for squamous non-small cell lung cancer, and as a second-line treatment for renal cell carcinoma.

“Pembrolizumab”, developed by MERCK, (trade name “KEYTRUDA”, also known as lambrolizumab, and MK-3475) is a humanized monoclonal IgG4 antibody directed against human cell surface receptor PD-1. Pembrolizumab is described, for example, in U.S. Pat. No. 8,900,587. It is for example indicated for the treatment of patients with unresectable or metastatic melanoma, as a single agent for the first-line treatment of patients with metastatic NSCLC whose tumors have high PD-L1 expression [(Tumor Proportion Score (TPS)≥50%)] as determined by an FDA-approved test, with no EGFR or ALK genomic tumor aberrations and also for the treatment of patients with recurrent or metastatic HNSCC with disease progression on or after platinum-containing chemotherapy.

“PDR-001”, developed by Novartis, is an intravenously administered anti-PD-1 antibody. In July 2017, Phase III trials for malignant melanoma, Phase II trials for nasopharyngeal cancer and for neuroendocrine tumors and Phase I/II trials for solid tumors and Phase I trials for hepatocellular carcinoma, lymphoma and colorectal cancer are ongoing.

“BGB-A317”, developed by BeiGene (China), currently is in a Phase Ia/Ib clinical trial in subjects with advanced tumors.

SHR-1210, developed by Jiangsu Hengrui Medicine, is another anti-PD-1 mAb and is in an open-label, multicenter, nonrandomized, dose escalation Phase I trial to evaluate its safety and tolerability.

JS001, developed by Shanghai Junshi Biosciences Co., Ltd., is a recombinant humanized monoclonal antibody. Phase II development for melanoma and bladder cancer, Phase I/II trial for gastric cancer, nasopharyngeal cancer, oesophageal cancer and head and neck cancer and Phase I development in breast cancer, lymphoma, urogenital cancer, renal cancer, neuroendocrine tumors and solid tumors are ongoing in July 2017.

STI-A1110 is a lead monoclonal antibody (MAb) against programmed cell death protein 1 (PD-1), under development by Sorrento Therapeutics using its G-MAB fully human antibody library platform, for the treatment of cancer. An initiation of clinical trial is expected in 2H 2017.

Pidilizumab (also known as BAT mAb, CT-011 and MDV9300) is a humanized antibody derived from the murine BAT-1 monoclonal antibody developed by Cure Tech. This antibody is currently in clinical trials for diffuse large B cell lymphoma, follicular lymphoma, and multiple myeloma, and has shown encouraging results and favorable toxicity.

“AMP-224”, developed by GlaxoSmithKline, is a PD-L2 lgG2a fusion protein that targets PD-1. The Phase I clinical study was finished in January 2014 in 44 patients with advanced cancer. Currently, this agent is in Phase II trials in combination with stereotactic body radiation therapy in patients with metastatic colorectal cancer.

“AMP-514” (also known as MEDI0680), developed by GlaxoSmithKline, is a PD-L2 fusion protein that targets PD-1. A Phase I multicenter open-label study to evaluate the safety tolerability and pharmacokinetics of AMP-514 in patients with advanced malignancies began in December 2013. Another Phase I study of AMP-514 in combination with MEDI4736 in patients with advanced malignancies currently is recruiting participants. In addition, there is a Phase Ib/II open-label study to evaluate the safety and/or efficacy of MEDI-551 in combination with AMP-514 in participants with relapsed or refractory aggressive B cell lymphomas who have failed one or two prior lines of therapy.

“Cemiplimab” (also known as REGN-2810) is a monoclonal antibody targeting PD-1 under development as a drug for the treatment of squamous cell skin cancer, myeloma, and lung cancer. In September 2018 it was approved by the US FDA for treating “patients with metastatic cutaneous squamous cell carcinoma (CSCC) or locally advanced CSCC who are not candidates for curative surgery or curative radiation”. Cemiplimab-rwlc will be marketed as Libtayo.

In one embodiment of present invention the PD-1 antagonist comprises

i) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or

ii) at least one heavy chain sequence that is at least 95%, at least 96%, at least 97%, at least 98%, at least 99%, or 100% identical to the sequence of SEQ ID NO.19; and/or

iii) at least one light chain sequence that is at least 95%, at least 96%, at least 97%, at least 98%, at least 99%, or 100% identical to the sequence of SEQ ID NO.20.

In a further embodiment the present invention provides for anti-ILDR2 antibody, a fragment or derivative thereof, a modified antibody format, or an antibody mimetic as defined herein for use in combination with a PD-1 antagonist as defined herein in the treatment of cancer, wherein at least one of the anti-ILDR2 antibody and the PD-1 antagonist is administered in simultaneous, separate, or sequential combination with one or more pharmaceutical agents.

The present invention furthermore provides for a novel combination comprising at least two components, component A and component B, wherein component A and component B are administered simultaneously, concurrently, separately or sequentially, and wherein component A is an anti-ILDR2 antibody, a fragment or derivative thereof, a modified antibody format, or an antibody mimetic as defined herein, and component B is a PD-1 antagonist as defined herein.

The present invention therefore provides in one aspect for novel combinations comprising at least two components, component A and component B, wherein component A is an anti-ILDR2 antibody, a fragment or derivative thereof, a modified antibody format, or an antibody mimetic, all of which having ILDR2 binding properties, and further comprising at least the three CDR heavy chain sequences according to SEQ ID NO.1, SEQ ID NO.2 and SEQ ID NO.3 and the three CDR light chain sequences according to SEQ ID NO.4, SEQ ID NO.5 and SEQ ID NO.6; and component B is a PD-1 antagonist.

In one embodiment of present invention component A is an anti-ILDR2 antibody, a fragment or derivative thereof, a modified antibody format, or an antibody mimetic, that comprises at least one heavy chain variable region sequence that is at least 95%, 96%, 97%, 98%, 99%, or 100% identical to the sequence of SEQ ID NO.7 and/or at least one light chain variable region sequence that is at least 95%, 96%, 97%, 98%, 99%, or 100% identical to the sequence of SEQ ID NO.8.

In a further embodiment of present invention component A is an anti-ILDR2 antibody, fragment or derivative thereof, a modified antibody format, or an antibody mimetic, comprises at least one heavy chain sequence that is at least 95%, 96%, 97%, 98%, 99%, or 100% identical to the sequence of SEQ ID NO.9; and/or at least one light chain sequence that is at least 95%, 96%, 97%, 98%, 99%, or 100% identical to the sequence of SEQ ID NO.10.

In a further embodiment component A is anti-ILDR2/no. 1 also referred to as aILDR2/no. 1. Antibody anti-ILDR2/no. 1 of present invention consists of a variable domain binding the extracellular domain of ILDR2 and a constant domain framework. The sequences of the heavy and light chain as well as the variable domains and the CDRs are disclosed in the sequences section as SEQ ID NOs.1-10. The antibody anti-ILDR2/no. 1 has first been characterized in patent application PCT/EP2018/082779, the content of which is herein incorporated by reference.

Another embodiment of present invention relates to the use of an anti-ILDR2 antibody, a fragment or derivative thereof, a modified antibody format, or an antibody mimetic as defined herein and a PD-1 antagonist as defined herein for the manufacture of a medicament for the treatment of cancer.

A further embodiment of present invention relates to a kit comprising an anti-ILDR2 antibody, a fragment or derivative thereof, a modified antibody format, or an antibody mimetic as described herein, a PD-1 antagonist as described herein and one or more further pharmaceutical agents.

### Therapeutic Methods

Therapeutic methods involve administering to a subject in need of treatment a therapeutically effective amount of an antibody or an antigen-binding fragment thereof or a variant thereof contemplated by the invention. A “therapeutically effective” amount hereby is defined as the amount of an antibody or antigen-binding fragment that is of sufficient quantity, either as a single dose or according to a multiple dose regimen, alone or in combination with other agents, to lead to the alleviation of an adverse condition, yet which amount is toxicologically tolerable. The subject may be a human or non-human animal (e.g., rabbit, rat, mouse, dog, monkey or other lower-order primate).

In one embodiment of present invention component A and component B are administered simultaneously, concurrently, separately or sequentially.

A further embodiment of present invention relates to the combination as described herein for use as a medicament for the treatment of cancer.

A further embodiment relates to the combination as described herein for use in the treatment or prophylaxis of a neoplastic disease, such as cancer, or an immune disease or disorder, wherein the combination is administered in one or more therapeutically efficient dosages.

Another embodiment of present invention relates to a method for treating a patient suffering from a neoplastic disease, such as cancer, comprising administering to said patient a combination as described herein in one or more therapeutically efficient dosages, wherein component A and component B are administered simultaneously, concurrently, separately or sequentially.

Another embodiment of present invention relates to a method for treating a patient suffering from a neoplastic disease, such as cancer, comprising administering to said patient an anti-ILDR2 antibody, a fragment or derivative thereof, a modified antibody format, or an antibody mimetic for the use as described herein and a PD-1 antagonist as defined herein, in one or more therapeutically efficient dosages, wherein the anti-ILDR2 antibody and the PD-1 antagonist are administered simultaneously, concurrently, separately or sequentially.

Disorders and conditions suitable for treatment with a composition of the present inventions can be, but are not limited to solid tumors, such as for example cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

Tumors of the digestive tract include, but are not limited to anal, colon, colorectal, esophageal, gallbladder, gastric, pancreatic, rectal, small-intestine, and salivary gland cancers.

Examples of esophageal cancer include, but are not limited to esophageal cell carcinomas and Adenocarcinomas, as well as squamous cell carcinomas, Leiomyosarcoma, Malignant melanoma, rhabdomyosarcoma and Lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumors.

Examples of breast cancer include, but are not limited to triple negative breast cancer, invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, glioblastoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal and vulvar cancer, as well as sarcoma of the uterus.

Examples of ovarian cancer include, but are not limited to serous tumour, endometrioid tumor, mucinous cystadenocarcinoma, granulosa cell tumor, Sertoli-Leydig cell tumor and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumors of the urinary tract include, but are not limited to bladder, penile, kidney, renal pelvis, ureter, urethral, and hereditary and sporadic papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to transitional cell carcinoma, squamous cell carcinoma, adenocarcinoma, sarcoma and small cell carcinoma.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct car¬cinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to squamous cell cancer of the head and neck, laryngeal, hypopharyngeal, nasopharyngeal, oropharyngeal cancer, salivary gland cancer, lip and oral cavity cancer, and squamous cell cancer.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′→3′.

1. Tumor Mouse Models

The CT26 tumor model was used in in vivo experiments. CT26 is an N-nitroso-N-methylurethane-(NNMU) induced, undifferentiated colon carcinoma cell line.

2. Antibody Generation

Antibodies against ILDR2 were generated by phage display. Briefly, panning reactions were carried out in solution using streptavidin-coated magnetic beads to capture the biotinylated antigens. Beads were recovered using a magnetic rack (Promega). All phage panning experiments used the XOMA031 human fab antibody phage display library (XOMA Corporation, Berkeley, Calif.) blocked with 5% skim milk.

Proteins required for phage display were biotinylated using a Sulfo-NHS-LC-Biotin kit (Pierce). Free biotin was removed from the reactions by dialysis against the appropriate buffer. The biotin labelled proteins included ILDR2-HM and the ECD of a control antigen fused to the same mouse IgG₂ₐ sequence. The control antigen was used for depletion steps in panning experiments. It was necessary to remove unwanted binders to streptavidin beads and the mouse IgG₂ₐ Fc domain during the panning process. To achieve this, streptavidin beads were coupled with the control antigens. A phage aliquot was then mixed with these ‘depletion’ beads and incubated at room temperature (RT) for 30 mins. The depletion beads were then discarded. For selection of specific binders to ILDR2-HM, the blocked and depleted phage library was mixed with magnetic beads coupled to biotinylated ILDR2-HM. Reactions were incubated at RT for 1-2 hrs and non-specific phage were removed by washing with PBS-T and PBS. After washing, bound phage were eluted by incubation with 100 mM triethylamine (EMD) and the eluate was neutralized by adding Tris-HCl pH 8.0 (Teknova).The resulting E. coli lawns were scraped and re-suspended in liquid growth media. A small aliquot of re-suspended cells was inoculated into a 100 mL culture (2YT with and ampicillin) and grown at 37° C. until the OD at 600 nM reached 0.5. This culture was infected with M13K07 helper phage (New England Biolabs) and kanamycin was added (selection antibiotic for M13K07). The culture was then maintained at 25° C. to allow phage packaging. An aliquot of the culture supernatant was carried over for either a subsequent round of panning or fab binding screens. Second and later rounds were conducted the same way, except that the rescued phage supernatant from the previous round was used in place of the phage library. The phage eluate was infected into TG1 E. coli, which transformed the cells with the XOMA031 phagemid. Transformed cells were then spread on selective agar plates (ampicillin) and incubated overnight at 37° C.The XOMA031 library is based on phagemid constructs that also function as IPTG inducible fab expression vectors. Eluted phage pools from panning round 3 were diluted and infected into TG1 E. coli cells (Lucigen) so that single colonies were generated when spread on an agar plate. Individual clones were grown in 1 mL cultures (2YT with glucose and ampicillin) and protein expression was induced by adding IPTG (Teknova). Expression cultures were incubated overnight at 25° C. Fab proteins secreted into the E. coli periplasm were then extracted for analysis. Each plate of samples also included duplicate ‘blank PPE’ wells to serve as negative controls. These were created from non-inoculated cultures processed the same way as the fab PPEs. FACS analyses were used to identify fabs with affinity for ILDR2. Individual fab PPEs were tested for binding to HEK-293T cells over-expressing human ILDR2 (293T-huILDR2 cells). All analyses included negative control HEK-293T cells mock transfected with an ‘empty vector’ control plasmid (293T-EV cells). Reagent preparation and wash steps were carried out in FACS buffer (PBS with 1% BSA). Fab and blank PPEs were mixed with an aliquot of cells, incubated for 1 hr at 4° C. and then washed with FACS buffer. Cells were then mixed with an anti-C-myc primary antibody (Roche). After the same incubation and wash step cells were stained with an anti-mouse IgG Fc AlexaFlour-647 antibody (Jackson Immunoresearch). After a final incubation and wash cells were fixed in 4% paraformaldehyde made up in FACS buffer. Samples were read on a HTFC screening system (Intellicyt). Data was analyzed using FCS Express (De Novo Software, CA, USA) or FloJo (De Novo Software, CA, USA). Based on these results, five binders were chosen for further analysis and reformatted into full length IgGs.

The antibody aILDR2/no. 1 of present invention consists of a variable domain binding the extracellular domain of ILDR2 and a constant domain framework. The sequences of the heavy and light chain as well as the variable domains and the CDRs are disclosed in the sequences section as SEQ ID NOs.1-10. The antibody aILDR2/no. 1 has first been characterized in patent application PCT/EP2018/082779.

Antibodies aPD-1 and aILDR2/no. 1 applied in the CT26 tumor in vivo experiment are controlled by isotype controls. The aILDR2/no. 1 antibody consists of a variable domain binding the extracellular domain of ILDR2 and a constant domain framework, and is controlled in in vivo experiments by a human IgG2 isotype control. The aPD-1 antibody consists of a variable domain binding the extracellular domain of PD-1 and a constant domain framework, and is controlled in in vivo experiments by a rat IgG2a isotype control.

TABLE 1
Antibodies used in the present study

| Alias Name | Details |
|---|---|
| aILDR2/no.1 | anti-ILDR2 antibody according to present |
| | invention: heavy and light chain, variable |
| | domains and CDRs as disclosed below as |
| | SEQ ID NOs. 1-10 |
| aPD-1 | RMP1-14 monoclonal antibody which reacts |
| | with mouse PD-1 (programmed death-1) |
| | also known as CD279 |
| hIgG2 isotype control | TPP-75 as disclosed in SEQ ID NOs. 31 |
| for aILDR2/no. 1 | and 32 |
| rIgG2a isotype | In VivoPlus rat IgG2a isotype control, anti- |
| control for aPD-1 | trinitrophenol, clone 2A3, catalog #BP0089 |
| | (BioXcell) |

3. Therapeutic, synergistic efficacy of aILDR2/no. 1 with aPD-1 in the CT26 tumor model

Nine weeks old female Balb/cAnN mice (body weight 18-22 g) from Charles River Deutschland, Sulzfeld were used for the CT26 tumor model. The experiment was initiated after an acclimatization period of 6 days. Animals were kept in a 12-hour light/dark cycle. Food and water was available ad libitum. Housing temperature was maintained at 21° C. Mice (n=12 per group) were s.c. inoculated with 5×10⁵ CT26 tumor cells into the left flank and assigned to experimental groups by stratified randomization (method for partitioning of the mice to groups with equal distribution of tumor size) on day 5 after tumor inoculation. At treatment initiation, animals were marked and each cage was labeled with the cage number, study number and the number of animals per cage.

Adjustment for in vivo administration with an application volume of 5 ml/kg was achieved by dilution of the stock solution in DPBS without Ca2+, Mg2+, pH 7.4 (Biochrom). aPD-1 was dosed i.p. at 10 mg/kg q3d×5 and aILDR2/no. 1 was dosed i.p. at 10 mg/kg q3d×5, all treatments starting on day 5 (d5). The experimental conditions are shown in the following table:

| | | | | | Appli- | Treat- |
|---|---|---|---|---|---|---|
| | N/ | | | | cation | ment |
| Group | group | Compound | Dose | route | volume | schedule |
| (1) Isotype | 12 | Isotype hIgG2 | 10 mg/kg | i.p. | 5 ml/kg | Q3D |
| control | | Isotype rIgG2a | 10 mg/kg | i.p. | 5 ml/kg | Q3D |
| (2) aILDR2 | 12 | aILDR2 | 10 mg/kg | i.p. | 5 ml/kg | Q3D |
| | | Isotype rIgG2a | 10 mg/kg | i.p. | 5 ml/kg | Q3D |
| (3) aPD-1 | 12 | aPD-1 | 10 mg/kg | i.p. | 5 ml/kg | Q3D |
| | | Isotype hIgG2 | 10 mg/kg | i.p. | 5 ml/kg | Q3D |
| (4) aILDR2 + | 12 | aILDR2 | 10 mg/kg | i.p. | 5 ml/kg | Q3D |
| aPD-1 | | aPD-1 | 10 mg/kg | i.p. | 5 ml/kg | Q3D |

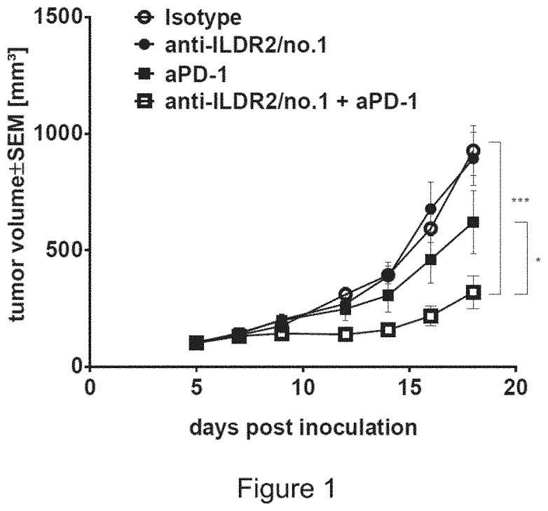

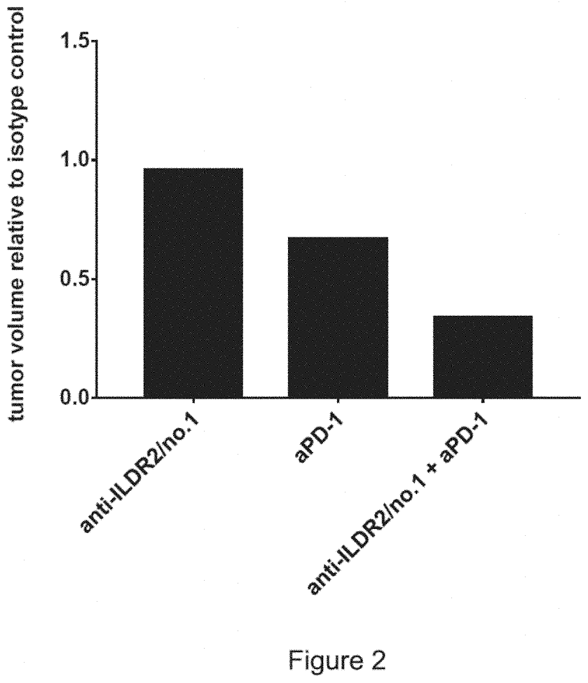

As can be seen in FIGS. 1 and 2 a combination of aPD-1 and aILDR2/no. 1 synergistically delayed tumor growth statistically significant compared to isotype control and compared to aILDR2/no. 1 or aPD-1 monotherapy.

### Sequences

The sequences shown in the following table are referred to herein. In case there is an ambiguity between this table and the WIPO standard sequence listing that forms part of the present specification and its disclosure, the sequences and qualifiers in this table shall be deemed the correct ones.

| SEQ ID | | | |
|---|---|---|---|
| NO. | Antibody | Region | Sequence |
| 1 | anti-ILDR2/no.1 | HCDR1 | SYAIS |
| 2 | anti-ILDR2/no.1 | HCDR2 | GIIPILGIANYAQKFQG |
| 3 | anti-ILDR2/no.1 | HCDR3 | ARGRLPYGDFWDS |
| 4 | anti-ILDR2/no.1 | LCDR1 | RSSQSLLYSNGYNYLD |
| 5 | anti-ILDR2/no.1 | LCDR2 | LGSNRAS |
| 6 | anti-ILDR2/no.1 | LCDR3 | MQALQTPLT |
| 7 | anti-ILDR2/no.1 | heavy | QVQLVQSGAEVKKPGSSVKVSCKASGGTFSSYAISWVR |
| | | chain VD | QAPGQGLEWMGGIIPILGIANYAQKFQGRVTITADKSTST |
| | | | AYMELSSLRSEDTAVYYCARGRLPYGDFWDSWGQGTL |
| | | | VTVSS |
| 8 | anti-ILDR2/no.1 | light | DIVMTQSPLSLPVTPGEPASISCRSSQSLLYSNGYNYLDW |
| | | chain VD | YLQKPGQSPQLLIYLGSNRASGVPDRFSGSGSGTDFTLKI |
| | | | SRVEAEDVGVYYCMQALQTPLTFGGGTKLEIR |
| 9 | anti-ILDR2/no.1 | heavy | QVQLVQSGAEVKKPGSSVKVSCKASGGTFSSYAISWVR |
| | | chain | QAPGQGLEWMGGIIPILGIANYAQKFQGRVTITADKSTST |
| | | | AYMELSSLRSEDTAVYYCARGRLPYGDFWDSWGQGTL |
| | | | VTVSSASTKGPSVFPLAPCSRSTSESTAALGCLVKDYFPE |
| | | | PVTVSWNSGALTSGVHTFPAVLQSSGLYSLSSVVTVPSS |
| | | | NFGTQTYTCNVDHKPSNTKVDKTVERKCCVECPPCPAPP |
| | | | VAGPSVFLFPPKPKDTLMISRTPEVTCVVVDVSHEDPEV |
| | | | QFNWYVDGVEVHNAKTKPREEQFNSTFRVVSVLTVVHQ |
| | | | DWLNGKEYKCKVSNKGLPAPIEKTISKTKGQPREPQVYT |
| | | | LPPSREEMTKNQVSLTCLVKGFYPSDIAVEWESNGQPEN |
| | | | NYKTTPPMLDSDGSFFLYSKLTVDKSRWQQGNVFSCSV |
| | | | MHEALHNHYTQKSLSLSPG |
| 10 | anti-ILDR2/no.1 | light | DIVMTQSPLSLPVTPGEPASISCRSSQSLLYSNGYNYLDW |
| | | chain | YLQKPGQSPQLLIYLGSNRASGVPDRFSGSGSGTDFTLKI |
| | | | SRVEAEDVGVYYCMQALQTPLTFGGGTKLEIRRTVAAPS |
| | | | VFIFPPSDEQLKSGTASVVCLLNNFYPREAKVQWKVDNA |
| | | | LQSGNSQESVTEQDSKDSTYSLSSTLTLSKADYEKHKVY |
| | | | ACEVTHQGLSSPVTKSFNRGEC |
| 11 | Pembrolizumab | VH | QVQLVQSGVEVKKPGASVKVSCKASGYTFTNYYMYWV |
| | | | RQAPGQGLEWMGGINPSNGGTNFNEKFKNRVTLTTDSS |
| | | | TTTAYMELKSLQFDDTAVYYCARRDYRFDMGFDYWGQ |
| | | | GTTVTVSS |
| 12 | Pembrolizumab | HCDR1 | NYYMY |
| 13 | Pembrolizumab | HCDR2 | GINPSNGGTNFNEKFKN |
| 14 | Pembrolizumab | HCDR3 | RDYRFDMGFDY |
| 15 | Pembrolizumab | VL | EIVLTQSPATLSLSPGERATLSCRASKGVSTSGYSYLHWY |
| | | | QQKPGQAPRLLIYLASYLESGVPARFSGSGSGTDFTLTISS |
| | | | LEPEDFAVYYCQHSRDLPLTFGGGTKVEIK |
| 16 | Pembrolizumab | LCDR1 | RASKGVSTSGYSYLH |
| 17 | Pembrolizumab | LCDR2 | LASYLES |
| 18 | Pembrolizumab | LCDR3 | QHSRDLPLT |
| 19 | Pembrolizumab | Heavy | QVQLVQSGVEVKKPGASVKVSCKASGYTFTNYYMYWV |
| | | Chain | RQAPGQGLEWMGGINPSNGGTNFNEKFKNRVTLTTDSS |
| | | | TTTAYMELKSLQFDDTAVYYCARRDYRFDMGFDYWGQ |
| | | | GTTVTVSSASTKGPSVFPLAPCSRSTSESTAALGCLVKDY |
| | | | FPEPVTVSWNSGALTSGVHTFPAVLQSSGLYSLSSVVTV |
| | | | PSSSLGTKTYTCNVDHKPSNTKVDKRVESKYGPPCPPCP |
| | | | APEFLGGPSVFLFPPKPKDTLMISRTPEVTCVVVDVSQED |
| | | | PEVQFNWYVDGVEVHNAKTKPREEQFNSTYRVVSVLTV |
| | | | LHQDWLNGKEYKCKVSNKGLPSSIEKTISKAKGQPREPQ |
| | | | VYTLPPSQEEMTKNQVSLTCLVKGFYPSDIAVEWESNGQ |
| | | | PENNYKTTPPVLDSDGSFFLYSRLTVDKSRWQEGNVFSC |
| | | | SVMHEALHNHYTQKSLSLSLGK |
| 20 | Pembrolizumab | Light | EIVLTQSPATLSLSPGERATLSCRASKGVSTSGYSYLHWY |
| | | Chain | QQKPGQAPRLLIYLASYLESGVPARFSGSGSGTDFTLTISS |
| | | | LEPEDFAVYYCQHSRDLPLTFGGGTKVEIKRTVAAPSVFI |
| | | | FPPSDEQLKSGTASVVCLLNNFYPREAKVQWKVDNALQ |
| | | | SGNSQESVTEQDSKDSTYSLSSTLTLSKADYEKHKVYAC |
| | | | EVTHQGLSSPVTKSFNRGEC |
| 21 | Nivolumab | VH | QVQLVESGGGVVQPGRSLRLDCKASGITFSNSGMEIWVR |
| | | | QAPGKGLEWVAVIWYDGSKRYYADSVKGRFTISRDNSK |
| | | | NTLFLQMNSLRAEDTAVYYCATNDDYWGQGTLVTVSS |
| 22 | Nivolumab | HCDR1 | NSGMH |
| 23 | Nivolumab | HCDR2 | VIWYDGSKRYYADSVKG |
| 24 | Nivolumab | HCDR3 | NDDY |
| 25 | Nivolumab | VL | EIVLTQSPATLSLSPGERATLSCRASQSVSSYLAWYQQKP |
| | | | GQAPRLLIYDASNRATGIPARFSGSGSGTDFTLTISSLEPE |
| | | | DFAVYYCQQSSNWPRTFGQGTKVEIK |
| 26 | Nivolumab | LCDR1 | RASQSVSSYLA |
| 27 | Nivolumab | LCDR2 | DASNRAT |
| 28 | Nivolumab | LCDR3 | QQSSNWPRT |
| 29 | Nivolumab | Heavy | QVQLVESGGGVVQPGRSLRLDCKASGITFSNSGMEIWVR |
| | | Chain | QAPGKGLEWVAVIWYDGSKRYYADSVKGRFTISRDNSK |
| | | | NTLFLQMNSLRAEDTAVYYCATNDDYWGQGTLVTVSS |
| | | | ASTKGPSVFPLAPCSRSTSESTAALGCLVKDYFPEPVTVS |
| | | | WNSGALTSGVHTFPAVLQSSGLYSLSSVVTVPSSSLGTK |
| | | | TYTCNVDHKPSNTKVDKRVESKYGPPCPPCPAPEFLGGP |
| | | | SVFLFPPKPKDTLMISRTPEVTCVVVDVSQEDPEVQFNW |
| | | | YVDGVEVHNAKTKPREEQFNSTYRVVSVLTVLHQDWL |
| | | | NGKEYKCKVSNKGLPSSIEKTISKAKGQPREPQVYTLPPS |
| | | | QEEMTKNQVSLTCLVKGFYPSDIAVEWESNGQPENNYK |
| | | | TTPPVLDSDGSFFLYSRLTVDKSRWQEGNVFSCSVMHEA |
| | | | LHNHYTQKSLSLSLGK |
| 30 | Nivolumab | Light | EIVLTQSPATLSLSPGERATLSCRASQSVSSYLAWYQQKP |
| | | Chain | GQAPRLLIYDASNRATGIPARFSGSGSGTDFTLTISSLEPE |
| | | | DFAVYYCQQSSNWPRTFGQGTKVEIKRTVAAPSVFIFPPS |
| | | | DEQLKSGTASVVCLLNNFYPREAKVQWKVDNALQSGNS |
| | | | QESVTEQDSKDSTYSLSSTLTLSKADYEKHKVYACEVTH |
| | | | QGLSSPVTKSFNRGEC |
| 31 | TPP-75 | Heavy | QVELLESGGGLVQPGGSLRLSCAASGFTFSSYAMSWVR |
| | Isotype control | Chain | QAPGKGLEWVSAISGSGGSTYYADSVKGRFTISRDNSKN |
| | for aILDR2 | | TLYLQMNSLRAEDTAVYYCARGVGKAHRFGVVPRGGM |
| | | | DVWGQGTLVTVSSASTKGPSVFPLAPCSRSTSESTAALG |
| | | | CLVKDYFPEPVTVSWNSGALTSGVHTFPAVLQSSGLYSL |
| | | | SSVVTVPSSNFGTQTYTCNVDHKPSNTKVDKTVERKCC |
| | | | VECPPCPAPPVAGPSVFLFPPKPKDTLMISRTPEVTCVVV |
| | | | DVSHEDPEVQFNWYVDGVEVHNAKTKPREEQFNSTFRV |
| | | | VSVLTVVHQDWLNGKEYKCKVSNKGLPAPIEKTISKTK |
| | | | GQPREPQVYTLPPSREEMTKNQVSLTCLVKGFYPSDIAV |
| | | | EWESNGQPENNYKTTPPMLDSDGSFFLYSKLTVDKSRW |
| | | | QQGNVFSCSVMHEALHNHYTQKSLSLSPGK |
| 32 | TPP-75 | Light | DIVLTQPPSASGTPGQRVTISCSGSSSNIGSNTVNWYQQL |
| | Isotype control | Chain | PGTAPKLLIYGNSNRPSGVPDRFSGSKSGTSASLAISGLRS |
| | for aILDR2 | | EDEADYYCAAWDDSLNGVLFGGGTKLTVLGQPKAAPS |
| | | | VTLFPPSSEELQANKATLVCLISDFYPGAVTVAWKGDSS |
| | | | PVKAGVETTTPSKQSNNKYAASSYLSLTPEQWKSHRSYS |
| | | | CQVTHEGSTVEKTVAPTECS |

1. A method of treating cancer comprising administering an anti-ILDR2 antibody, a fragment or derivative thereof, a modified antibody format, or an antibody mimetic simultaneously, concurrently, separately or sequentially with an effective amount of a PD-1 antagonist in the treatment of cancer, wherein the anti-ILDR2 antibody, a fragment or derivative thereof, a modified antibody format, or an antibody mimetic further comprises at least the three CDR heavy chain sequences according to SEQ ID NO.1, SEQ ID NO.2 and SEQ ID NO.3 and the three CDR light chain sequences according to SEQ ID NO.4, SEQ ID NO.5 and SEQ ID NO.6.

2. The method according to claim 1, wherein the anti-ILDR2 antibody, fragment or derivative thereof, modified antibody format or antibody mimetic comprises
(i) at least one heavy chain variable region sequence that is at least 95%, at least 96%, at least 97%, at least 98%, at least 99%, or 100% identical to the sequence of SEQ ID NO.7, and/or
(ii) at least one light chain variable region sequence that is at least 95%, at least 96%, at least 97%, at least 98%, at least 99%, or 100% identical to the sequence of SEQ ID NO.8.

3. The method according to claim 1, wherein the anti-ILDR2 antibody, fragment or derivative thereof, modified antibody format or antibody mimetic comprises
(i) at least one heavy chain sequence that is at least 95%, at least 96%, at least 97%, at least 98%, at least 99%, or 100% identical to the sequence of SEQ ID NO.9; and/or
(ii) at least one light chain sequence that is at least 95%, at least 96%, at least 97%, at least 98%, at least 99%, or 100% identical to the sequence of SEQ ID NO.10.

4. The method according to claim 1, wherein the PD-1 antagonist is an antibody, a fragment or derivative thereof, a modified antibody format, or an antibody mimetic, all of which having PD-1 binding properties.

5. The method according to claim 1, wherein the PD-1 antagonist is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine).

6. The method according to claim 1, wherein the PD-1 antagonist is nivolumab (Opdivo, BMS-936558, MDX1106) or pembrolizumab (Keytruda, MK-3475, lambrolizumab), preferably pembrolizumab (Keytruda, MK-3475, lambrolizumab).

7. The method according to claim 1, wherein the PD-1 antagonist comprises
i) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
ii) at least one heavy chain sequence that is at least 95%, at least 96%, at least 97%, at least 98%, at least 99%, or 100% identical to the sequence of SEQ ID NO.19; and/or
iii) at least one light chain sequence that is at least 95%, at least 96%, at least 97%, at least 98%, at least 99%, or 100% identical to the sequence of SEQ ID NO.20.

8. The method according to claim 1, wherein at least one of the anti-ILDR2 antibody and the PD-1 antagonist is administered in simultaneous, separate, or sequential combination with one or more pharmaceutical agents.

9. A combination comprising at least two components, component A and component B, wherein component A and component B are administered simultaneously, concurrently, separately or sequentially, and wherein
i) component A is an anti-ILDR2 antibody, a fragment or derivative thereof, a modified antibody format, or an antibody mimetic as defined in claim 1; and
ii) component B is a PD-1 antagonist that:
a) is an antibody, a fragment or derivative thereof, a modified antibody format, or an antibody mimetic, all of which having PD-1 binding properties,
b) is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine),
c) is nivolumab (Opdivo, BMS-936558, MDX1106) or pembrolizumab (Keytruda, MK-3475, lambrolizumab), or
d) comprises:
1) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
2) at least one heavy chain sequence that is at least 95%, at least 96%, at least 97%, at least 98%, at least 99%, or 100% identical to the sequence of SEQ ID NO.19; and/or
3) at least one light chain sequence that is at least 95%, at least 96%, at least 97%, at least 98%, at least 99%, or 100% identical to the sequence of SEQ ID NO.20.

10. The combination according to claim 9 for use as a medicament.

11. The combination according to claim 9 for use in the treatment or prophylaxis of a neoplastic disease, such as cancer, or an immune disease or disorder, wherein the combination is administered in one or more therapeutically efficient dosages.

12-13. (canceled)

14. A kit comprising
i) an anti-ILDR2 antibody, a fragment or derivative thereof, a modified antibody format, or an antibody mimetic as defined in claim 1; and
ii) a PD-1 antagonist that:
a) is an antibody, a fragment or derivative thereof, a modified antibody format, or an antibody mimetic, all of which having PD-1 binding properties,
b) is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine),
c) is nivolumab (Opdivo, BMS-936558, MDX1106) or pembrolizumab (Keytruda, MK-3475, lambrolizumab), or
d) comprises:
1) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
2) at least one heavy chain sequence that is at least 95%, at least 96%, at least 97%, at least 98%, at least 99%, or 100% identical to the sequence of SEQ ID NO.19; and/or
3) at least one light chain sequence that is at least 95%, at least 96%, at least 97%, at least 98%, at least 99%, or 100% identical to the sequence of SEQ ID NO.20; and
iii) one or more further pharmaceutical agents.

15. A kit comprising
i) an anti-ILDR2 antibody, a fragment or derivative thereof, a modified antibody format, or an antibody mimetic as defined in claim 2; and
ii) a PD-1 antagonist that:
a) is an antibody, a fragment or derivative thereof, a modified antibody format, or an antibody mimetic, all of which having PD-1 binding properties,
b) is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine),
c) is nivolumab (Opdivo, BMS-936558, MDX1106) or pembrolizumab (Keytruda, MK-3475, lambrolizumab), or
d) comprises:
1) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
2) at least one heavy chain sequence that is at least 95%, at least 96%, at least 97%, at least 98%, at least 99%, or 100% identical to the sequence of SEQ ID NO.19; and/or
3) at least one light chain sequence that is at least 95%, at least 96%, at least 97%, at least 98%, at least 99%, or 100% identical to the sequence of SEQ ID NO.20; and
iii) one or more further pharmaceutical agents.

16. A kit comprising
i) an anti-ILDR2 antibody, a fragment or derivative thereof, a modified antibody format, or an antibody mimetic as defined in claim 3; and
ii) a PD-1 antagonist that:
a) is an antibody, a fragment or derivative thereof, a modified antibody format, or an antibody mimetic, all of which having PD-1 binding properties,
b) is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine),
c) is nivolumab (Opdivo, BMS-936558, MDX1106) or pembrolizumab (Keytruda, MK-3475, lambrolizumab), or
d) comprises:
1) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
2) at least one heavy chain sequence that is at least 95%, at least 96%, at least 97%, at least 98%, at least 99%, or 100% identical to the sequence of SEQ ID NO.19; and/or
3) at least one light chain sequence that is at least 95%, at least 96%, at least 97%, at least 98%, at least 99%, or 100% identical to the sequence of SEQ ID NO.20; and
iii) one or more further pharmaceutical agents.

17. A combination comprising at least two components, component A and component B, wherein component A and component B are administered simultaneously, concurrently, separately or sequentially, and wherein
i) component A is an anti-ILDR2 antibody, a fragment or derivative thereof, a modified antibody format, or an antibody mimetic as defined in claim 2; and
ii) component B is a PD-1 antagonist that:
a) is an antibody, a fragment or derivative thereof, a modified antibody format, or an antibody mimetic, all of which having PD-1 binding properties,
b) is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine),
c) is nivolumab (Opdivo, BMS-936558, MDX1106) or pembrolizumab (Keytruda, MK-3475, lambrolizumab), or
d) comprises:
1) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
2) at least one heavy chain sequence that is at least 95%, at least 96%, at least 97%, at least 98%, at least 99%, or 100% identical to the sequence of SEQ ID NO.19; and/or
3) at least one light chain sequence that is at least 95%, at least 96%, at least 97%, at least 98%, at least 99%, or 100% identical to the sequence of SEQ ID NO.20.

18. A combination comprising at least two components, component A and component B, wherein component A and component B are administered simultaneously, concurrently, separately or sequentially, and wherein
i) component A is an anti-ILDR2 antibody, a fragment or derivative thereof, a modified antibody format, or an antibody mimetic as defined in claim 3; and
ii) component B is a PD-1 antagonist that:
a) is an antibody, a fragment or derivative thereof, a modified antibody format, or an antibody mimetic, all of which having PD-1 binding properties,
b) is selected from the group consisting of nivolumab (Opdivo, BMS-936558, MDX1106), pembrolizumab (Keytruda, MK-3475, lambrolizumab), PDR-001 (Novartis), JS001 (Shanghai Junshi Biosciences), STI-A1110, pidilizumab (Cure Tech), AMP-224 (GlaxoSmithKline), AMP-514 (GlaxoSmithKline), cemiplimab (Regeneron and Sanofi), BGB-A317 (BeiGene, China), SHR-1210 (Jiangsu Hengrui Medicine),
c) is nivolumab (Opdivo, BMS-936558, MDX1106) or pembrolizumab (Keytruda, MK-3475, lambrolizumab), or
d) comprises:
1) at least the three CDR heavy chain sequences according to SEQ ID NO.12, SEQ ID NO.13 and SEQ ID NO.14 and the three CDR light chain sequences according to SEQ ID NO.16, SEQ ID NO.17 and SEQ ID NO.18; and/or
2) at least one heavy chain sequence that is at least 95%, at least 96%, at least 97%, at least 98%, at least 99%, or 100% identical to the sequence of SEQ ID NO.19; and/or
3) at least one light chain sequence that is at least 95%, at least 96%, at least 97%, at least 98%, at least 99%, or 100% identical to the sequence of SEQ ID NO.20.
